(19) United States
(12) Patent Application Publication
(10) Pub. No.: US 2025/0262509 A1
(43) Pub. Date: Aug. 21, 2025

(54) System and Method for Timing Personal Physical Activity

(71) Applicant: Oswald Leroy Skeete, Phoenix, AZ (US)

(72) Inventor: Oswald Leroy Skeete, Phoenix, AZ (US)

(21) Appl. No.: 19/180,773

(22) Filed: Apr. 16, 2025

Related U.S. Application Data
(63) Continuation-in-part of application No. 17/410,905, filed on Aug. 24, 2021, now Pat. No. 12,290,739.
(60) Provisional application No. 63/069,574, filed on Aug. 24, 2020.

(51) Int. Cl. A63B 71/06

(52) U.S. Cl. CPC A63B 71/0686; A63B 2220/12; A63B 2220/20; A63B 2220/40; A63B 2220/62

(57) ABSTRACT

A system and method for timing personal physical activity provides an athlete with the ability to self-time runs over specific distances from anywhere. This can be used for any event that is timed over a specified distance. A software application utilizes the internal hardware commonly provided by many smart-devices, such as global positioning system (GPS) modules and accelerometers, to assist the user in tracking and recording the time and distance of a walk/run. The software application is compatible with any smart-devices, such as smart-phones, smart-watches, and a variety of other smart-wearables. In addition, GPS is utilized to track and record the distance travelled by the user. The software application also uses a stopwatch-like mechanism in order to track and record the duration of an exercise. Furthermore, the GPS distance tracker and the stopwatch-like timer may be used together to create a pacemaker/pacesetter for the user.

The current application is a continuation-in-part (CIP) application of the U.S. non-provisional application Ser. No. 17/410,905 filed on Apr. 6, 2025. The U.S. non-provisional application Ser. No. 17/410,905 claims a priority to the U.S. Provisional Patent application Ser. No. 63/069,574 filed on Aug. 24, 2020.

### FIELD OF THE INVENTION

The present invention relates to automatically recording time taken to run, walk, or swim a specific distance set by the user. More specifically, the present invention is a method that allows the user to mimic an athletic event, such as a 100 m or 200 m dash, without utilizing a track, a defined start or finish line, a starter, or a time piece based on the starter's gun. The software application coupled with a smart-device like a phone or watch is used to activate and record the time and distance.

### BACKGROUND OF THE INVENTION

Traditionally, an athlete may go to a track to practice or run their event of interest. Tracks are generally laid out with specific markers that translate to specific distances, like 100 meters, 200 meters etc. An athlete may typically be timed over a certain distance by a coach using a stopwatch. In the case of a competition, there will often be a starter with a gun connected to a timing mechanism. Furthermore, when running or walking, an athlete can use a watch or smart-phone to keep track of the time and distance it took to complete a walk/run. In training or other such cases, however, athletes may not have access to a track, or a coach. After weeks, the same person may want to track the time and distance in order to quantitatively measure progress from the previous walk/run. One may use a stopwatch to track or time the walk or run but may not necessarily be able to track the distance unless they were on a track.

The present invention addresses this issue by giving the athlete the ability to self-time over a specific distance anywhere they choose. This can be used for any event that is timed over a specified distance. Therefore, an objective of the present invention is to provide a software application utilizing the internal hardware of many smart-devices, such as global positioning system (GPS) modules and accelerometers, to assist the user in tracking and recording the time and distance of a walk/run. The present invention uses a software application that is compatible with any smart-devices, such as smart-phones, smart-watches, and a variety of other smart-wearables. In addition, the present invention utilizes GPS to track and record the distance travelled by the user. The software application also uses a stopwatch-like mechanism in order to track and record the time ran by the user. Furthermore, the present invention utilizes the GPS distance tracker and the stopwatch-like timer together in order to create a pacemaker/pacesetter for the user, also known as a rabbit. The concept of a rabbit is well known and used in athletic completions (track and field meet).

### BRIEF DESCRIPTION OF THE DRAWINGS

FIG. 1 is a block diagram illustrating the system of the present invention.

FIG. 2 is a flowchart illustrating the overall process for the method of the present invention.

FIG. 3 is a flowchart illustrating a subprocess of selecting the desired activity distance.

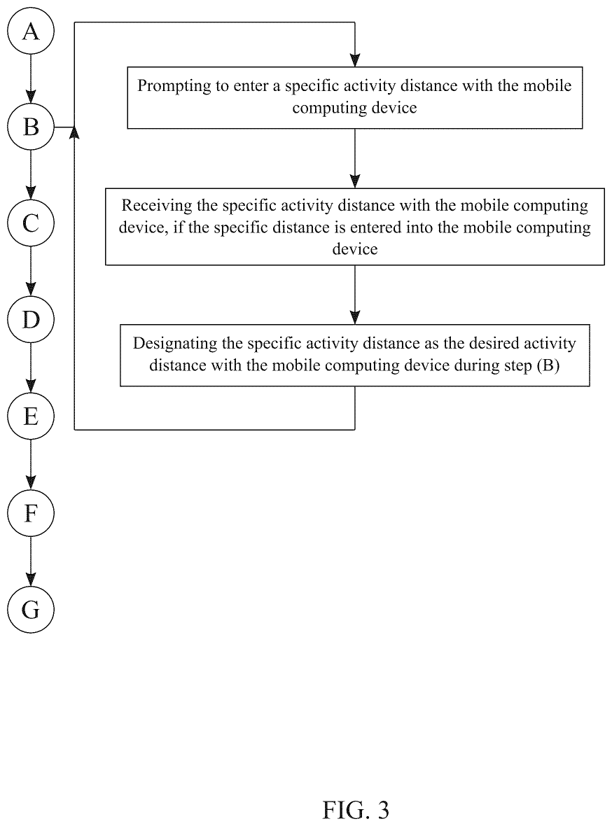

FIG. 4 is a flowchart illustrating a subprocess of beginning a countdown timer.

FIG. 5 is a flowchart illustrating a subprocess of specifying the countdown timer.

FIG. 6 is a flowchart illustrating a subprocess of detecting false starts.

FIG. 7 is a flowchart illustrating a subprocess of beginning timing based on user input.

FIG. 8 is a flowchart illustrating a subprocess of beginning timing based on accelerometer input from an unmoving start.

FIG. 9 is a flowchart illustrating a subprocess of logging a user's reaction time from the unmoving start.

FIG. 10 is a flowchart illustrating a subprocess of logging a user's acceleration from the unmoving start.

FIG. 11 is a flowchart illustrating a subprocess of beginning timing based on accelerometer input from a moving start.

FIG. 12 is a flowchart illustrating a subprocess of logging a user's reaction time from the unmoving start.

FIG. 13 is a flowchart illustrating a subprocess of logging a user's acceleration from the unmoving start.

FIG. 14 is a flowchart illustrating a subprocess of pausing the timer.

FIG. 15 is a flowchart illustrating a subprocess of resetting the timer.

FIG. 16 is a flowchart illustrating a subprocess of lapping the timer.

FIG. 17 is a flowchart illustrating a subprocess of utilizing location services.

FIG. 18 is a flowchart illustrating a subprocess of tracking a user's pre-timing distance.

FIG. 19 is a flowchart illustrating a subprocess of selecting the user's pre-timing distance.

FIG. 20 is a flowchart illustrating a subprocess of logging a lapsed time for the user's pre-timing distance.

FIG. 21 is a flowchart illustrating a subprocess of tracking a specific point along the desired activity distance.

FIG. 22 is a flowchart illustrating a subprocess of tracking biomechanical motion of a user's workout.

FIG. 23 is a flowchart illustrating a subprocess of tracking biological metric of a user's workout.

FIG. 24 is a flowchart illustrating a subprocess of viewing an athlete database.

### DETAILED DESCRIPTION OF THE INVENTION

All illustrations of the drawings are for the purpose of describing selected versions of the present invention and are not intended to limit the scope of the present invention.

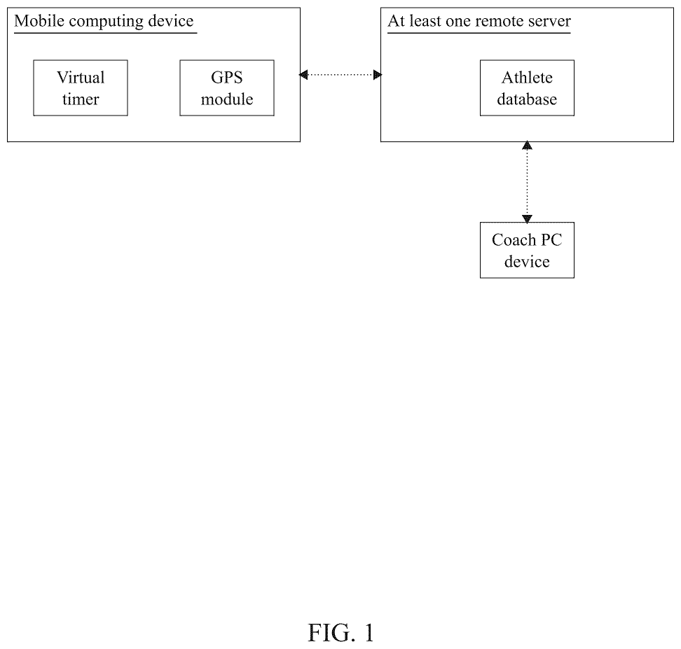

The present invention is a system and method for timing personal physical activity that allows an athlete to accurately time their physical activity without external assistance from a coach or other third party. The present invention accomplishes this by providing alternative mechanisms for determining the beginning and end of a workout or run. The system of the present invention includes a virtual timer managed by a mobile computing device, wherein the mobile computing device includes a global positioning system (GPS) module (Step A), as represented in FIG. 1. The virtual timer denotes an internal clock within the mobile computing device that allows for measurement of elapsed time. The mobile computing device may be any of standard phones, smart-phones, smart-wearables (e.g., smart-earbuds), laptops, personal computers, or other such devices capable of connecting wirelessly to the Internet and displaying relevant information to the user. The GPS module is the subsystem of the mobile computing device that enables tracking of the location of the mobile computing device.

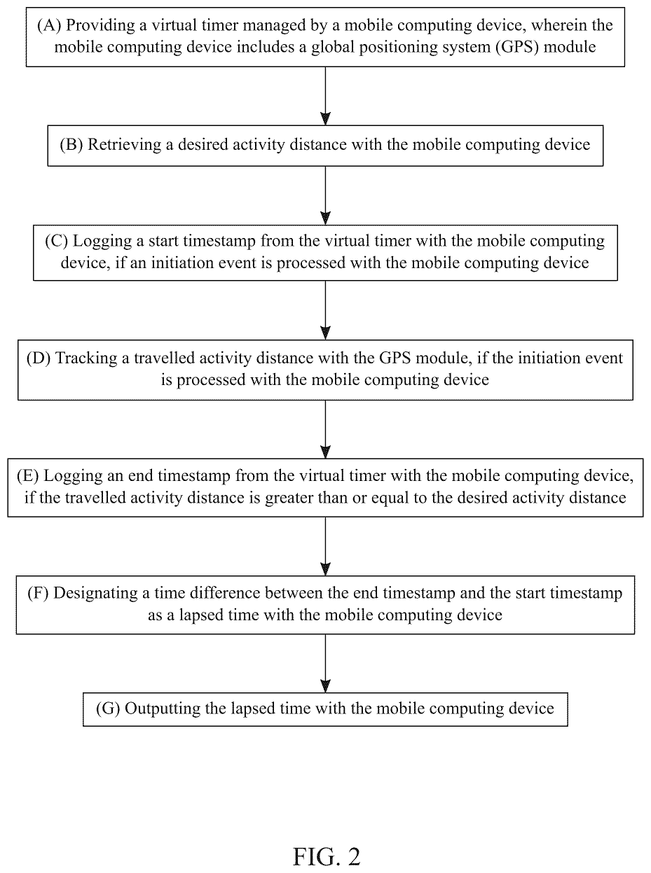

The overall process followed by the method of the present invention allows for effective and efficient tracking, calculating, and reporting of the amount of time that passes as the user traverses a given distance. A desired activity distance is retrieved with the mobile computing device (Step B), as represented in FIG. 2. The desired activity distance denotes a recorded user input representing the distance the user wishes to traverse during exercise. Next, a start timestamp from the virtual timer is logged with the mobile computing device, if an initiation event is processed with the mobile computing device (Step C). The start timestamp is the moment representing the beginning of the workout, or, in some cases, the beginning of the meaningful, timed portion of the workout. The initiation event is an occurrence that indicates the beginning of the timed portion of a workout. Subsequently, a travelled activity distance is tracked with the GPS module, if the initiation event is processed with the mobile computing device (Step D). specific activity distance relates to a specified number of feet, meters, miles, or other units of measurement, but is not limited to any particular geographic arrangement; the specific activity distance may ultimately be traversed on flat, rough, or other terrain. The specific activity distance may then be received with the mobile computing device, if the specific distance is entered into the mobile computing device. This arrangement ensures that the mobile computing device has access to relevant data necessary to complete time elapsed computation. Finally, the specific activity distance may be designated as the desired activity distance with the mobile computing device during Step B. Thus, the mobile computing device may utilize user input in determining the appropriate outputs to calculate and subsequently display to the user. An end timestamp from the virtual timer is logged with the mobile computing device (Step E), if the travelled activity distance is greater than or equal to the desired activity distance, and a time difference between the end timestamp and the start timestamp is then designated as an activity lapsed time with the mobile computing device (Step F). The overall process concludes by outputting the activity lapsed time with the mobile computing device (Step G). In a preferred embodiment, an athlete database is stored and managed by at least one remote server so that athletic metrics and/or other information about a user can be gathered and stored for future reference (i.e., a kind of cloud database). Thus, the desired activity distance, the start timestamp, the end timestamp, and the travelled activity distance are preferably amended into the athlete database with the remote server.

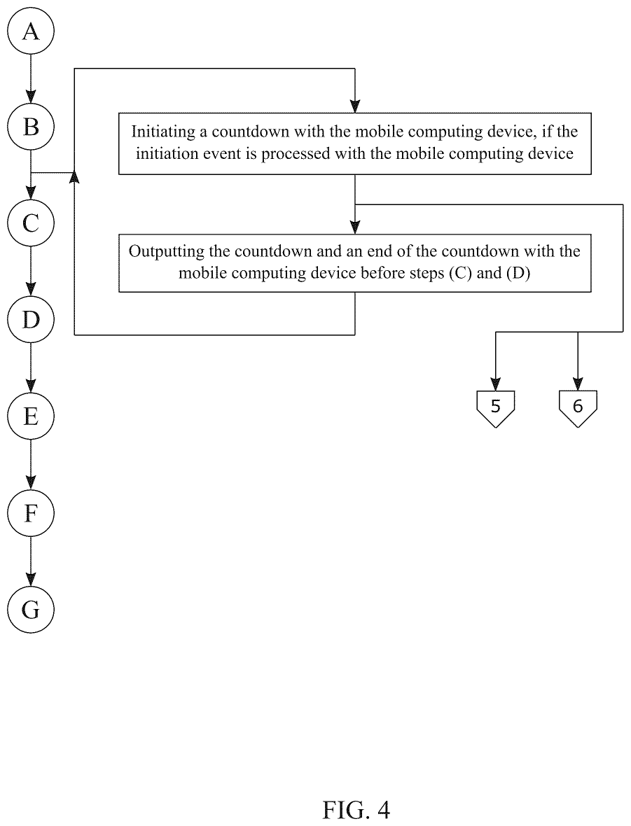

A user of the present invention may wish to start a workout upon completion of a countdown, thereby simulating race conditions. To achieve this, a countdown may be initiated with the mobile computing device, if the initiation event is processed with the mobile computing device, as represented in FIG. 4. The countdown is a periodic decrement of a numerical value to zero. The countdown and an end of the countdown may then be outputted with the mobile computing device before Steps C and D. Thus, a user may, in a preferred usage of the present invention, view the countdown before beginning exercise activity.

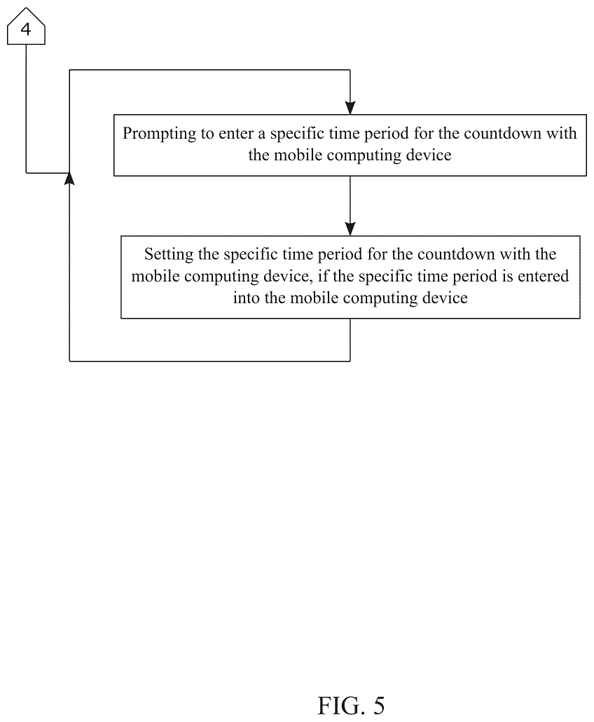

Furthermore, a user may benefit from specifying the length of a countdown in order to enable simulation of different race start conditions. To this end, a specific time period for the countdown may be prompted to be entered with the mobile computing device, as represented in FIG. 5. The specific time period may be listed in seconds, milliseconds, minutes, hours, or other units as desired by the user. The specific time period for the countdown may subsequently be set with the mobile computing device, if the specific time period is entered into the mobile computing device. Thus, the mobile computing device may begin the countdown at a desired starting time.

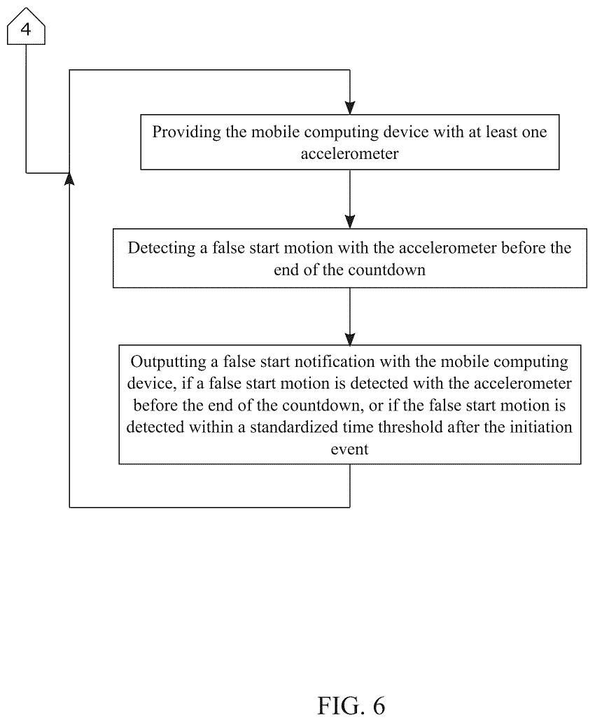

An athlete may wish to practice or test their ability to respond to a starting gun, thereby simulating races. To achieve this, the mobile computing device may be provided with an accelerometer, as represented in FIG. 6. The accelerometer is a sensor capable of detecting changes in the position of the mobile computing device. A false start motion may be detected with the accelerometer before the end of the countdown. The false start motion may be any amount of movement registered by the accelerometer before the end of the countdown. Alternatively, the false start motion may be detected within a standardized time threshold after the initiation event. For example, as it is common for racers to be required to wait until one-tenth of a second after the starting gun during many races, the detection of a false start motion may be delayed after the countdown as desired. Moreover, the standardized time threshold is preferably set by an Olympic committee to be up to 100 milliseconds. Finally, a false start notification may be outputted with the mobile computing device. The false start notification may be any combination of visual, audio, or tactile alerts. Thus, the user may become aware of the false start and may subsequently practice reaction timing. In the preferred embodiment, the false start motion is further amended into the athlete database with the remote server.

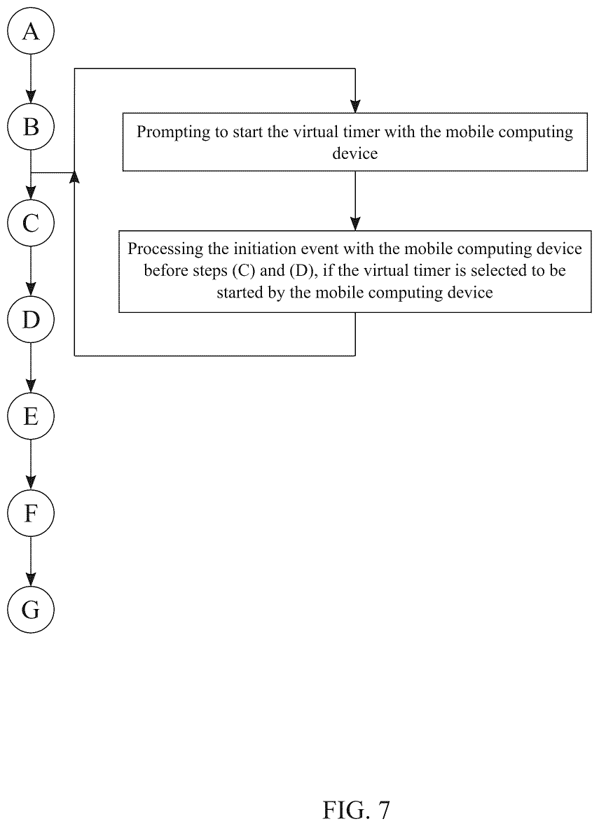

The user may wish to utilize the virtual timer as a regular timer. To enable this, the virtual timer may be prompted with the mobile computing device, as represented in FIG. 7. This enables the mobile computing device to collect and process user preferences prior to starting the virtual timer. Subsequently, the initiation event may be processed with the mobile computing device before Steps C and D, if the virtual timer is selected to be started by the mobile computing device. In this way, the virtual timer may be started according to the desire expressed by the user through accepting the prompt.

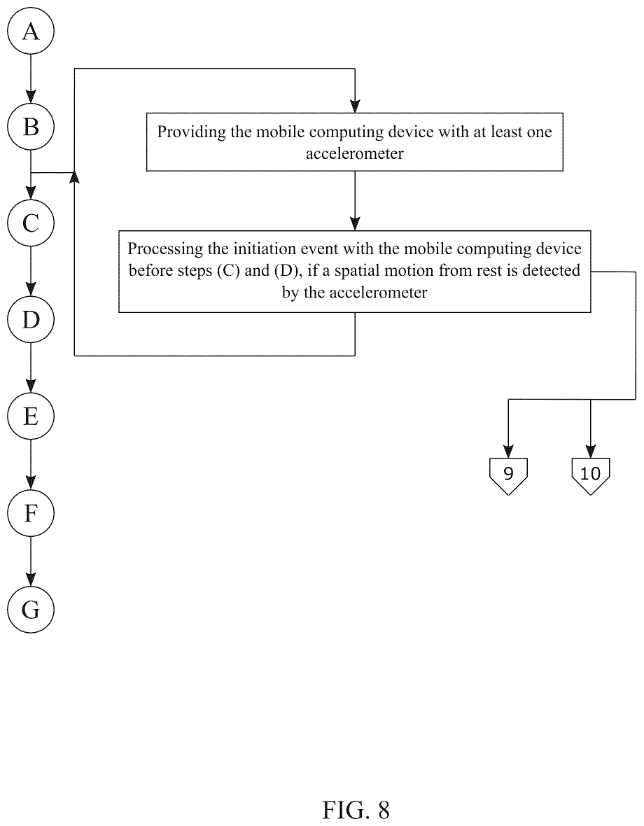

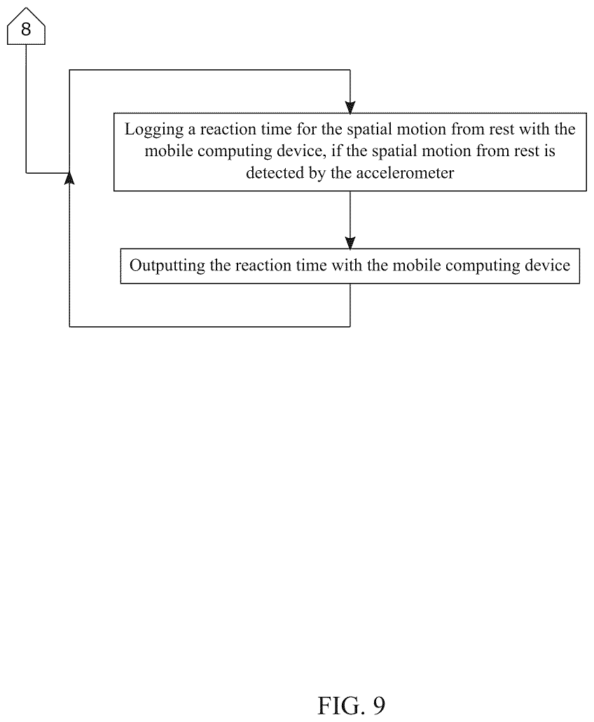

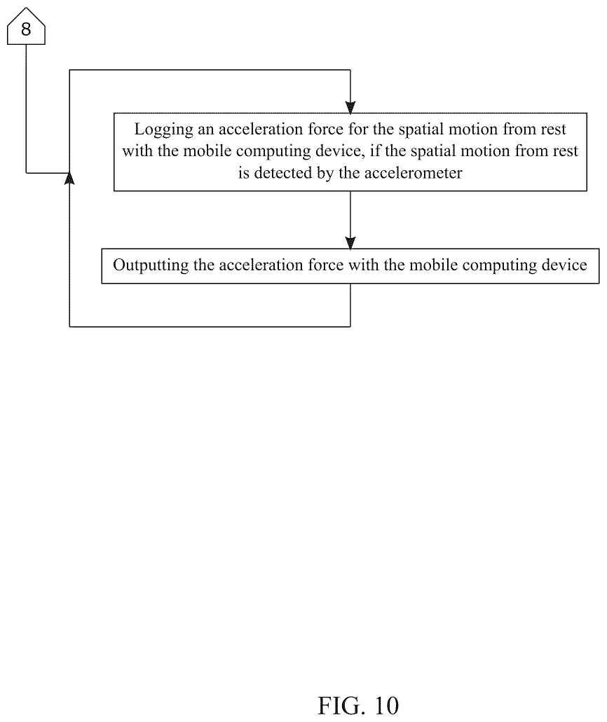

The virtual timer is further equipped to begin timing an activity in response to the motion of the user. To enable this, the mobile computing device may be provided with at least one accelerometer, as represented in FIG. 8. The accelerometer is a sensor capable of detecting changes in the position of the mobile computing device. The initiation event may then be processed with the mobile computing device before Steps C and D, if a spatial motion from rest is detected by the accelerometer. The spatial motion from rest may be movement along any plane or vector. Thus, the virtual timer can begin tracking a workout or exercise activity as a user begins moving. Moreover, as can be seen FIG. 9, in order for a user to view their reaction time during this kind of start, a reaction time for the spatial motion from rest is logged with the mobile computing device, if the spatial motion from rest is detected by an accelerometer of the mobile computing device, which eventually allows the reaction time to be outputted with the mobile computing device. Furthermore, as can be seen FIG. 10, in order for a user to view their acceleration during this kind of start, an acceleration force for the spatial motion from rest is logged with the mobile computing device, if the spatial motion from rest is detected by an accelerometer of the mobile computing device, which eventually allows the acceleration force to be outputted with the mobile computing device. In the preferred embodiment, the reaction time and the acceleration force for the spatial motion from rest are further amended into the athlete database with the remote server.

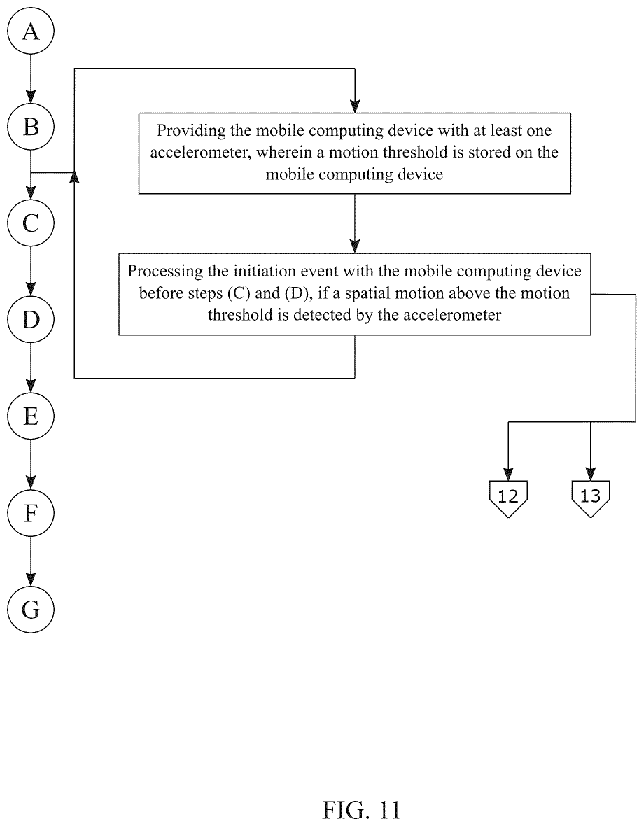

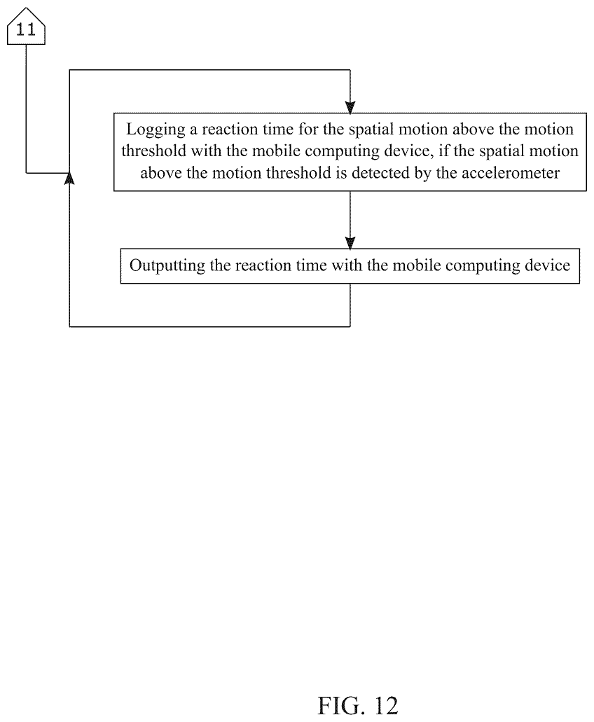

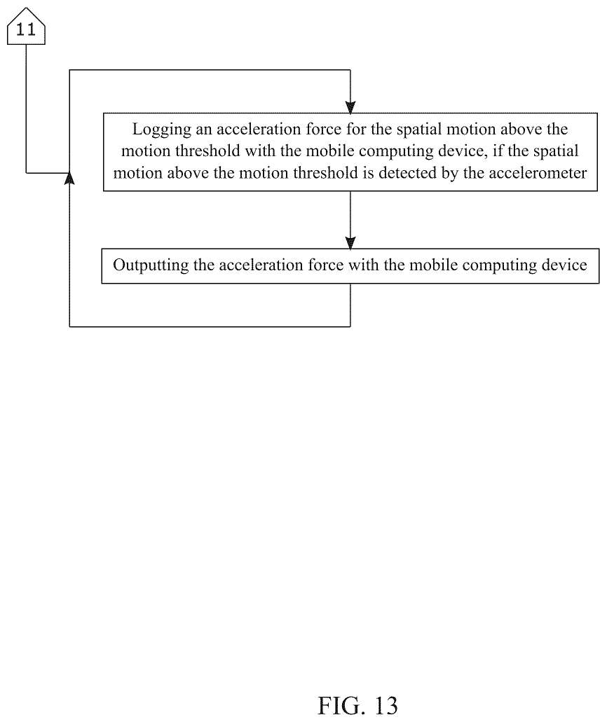

It may be further desirable to prevent the mobile computing device from activating until an appropriate amount of movement is detected. To this end, the mobile computing device may be provided with at least one accelerometer, wherein a motion threshold is stored on the mobile computing device, as represented in FIG. 11. The motion threshold denotes a minimum value requirement that must be registered by the accelerometer before a signal can be sent. The initiation event may then be processed with the mobile computing device before Steps C and D, if a spatial motion above the motion threshold is detected by the accelerometer. This arrangement allows a user to get up to a desired speed and subsequently time the duration of high-speed movement during a workout. Moreover, as can be seen FIG. 12, in order for a user to view their reaction time during this kind of start, a reaction time for the spatial motion above the motion threshold is logged with the mobile computing device, if the spatial motion above the motion threshold is detected by an accelerometer of the mobile computing device, which eventually allows the reaction time to be outputted with the mobile computing device. Furthermore, as can be seen FIG. 13, in order for a user to view their acceleration during this kind of start, an acceleration force for the spatial motion above the motion threshold is logged with the mobile computing device, if the spatial motion above the motion threshold is detected by an accelerometer of the mobile computing device, which eventually allows the acceleration force to be outputted with the mobile computing device. In the preferred embodiment, the reaction time and the acceleration force for the spatial motion above the motion threshold are further amended into the athlete database with the remote server.

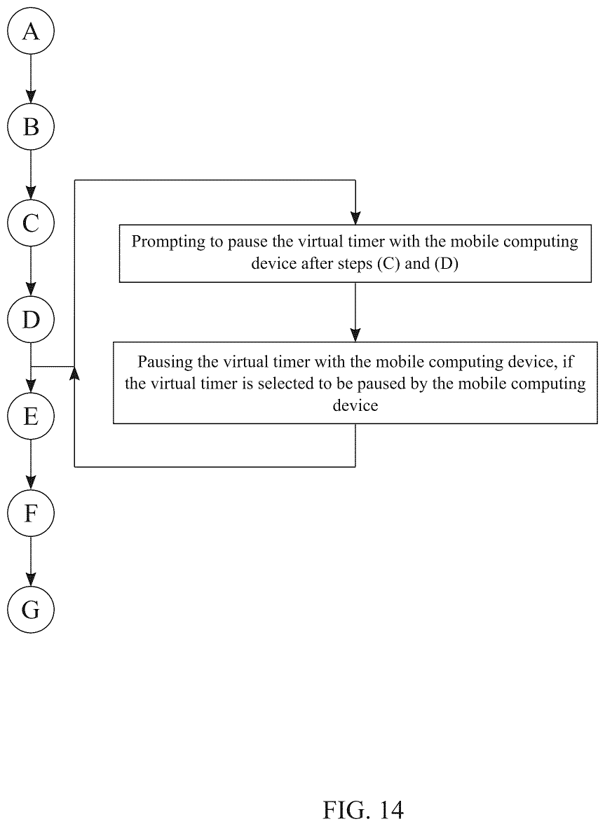

A user may wish to pause a workout to take a break, or to enable different types of interval training. To allow for this, the user may be prompted to pause the virtual timer with the mobile computing device after Steps C and D, as represented in FIG. 14. This arrangement allows a user to decide whether to pause the timer during a workout or exercise session. The virtual timer may then be paused with the mobile computing device, if the virtual timer is selected to be paused by the mobile computing device. Thus, the user may prevent the virtual timer from counting during undesirable portions of a workout and may resume timing as desired.

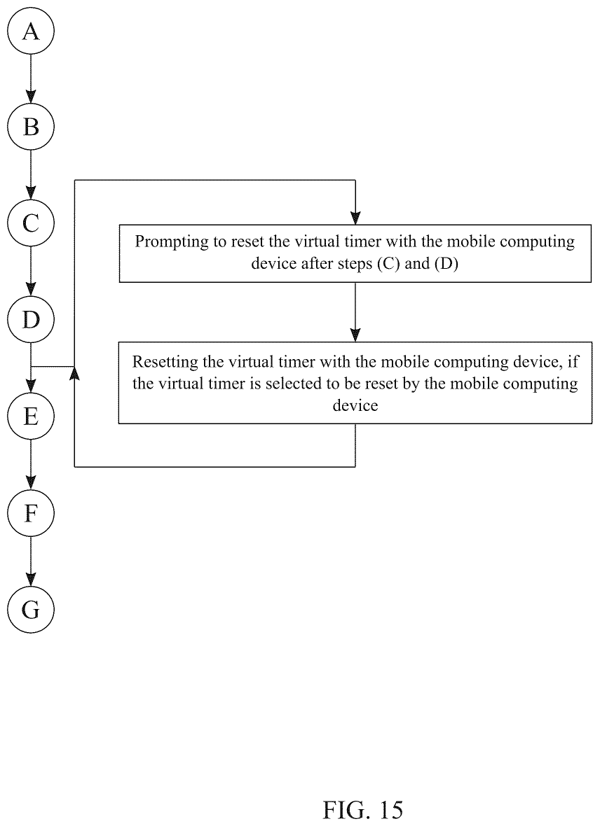

It may further be desirable to allow a user to reset a workout, thus providing a fresh start point for a new workout. To this end, the user may be prompted to reset the virtual timer with the mobile computing device after Steps C and D, as represented in FIG. 15. This arrangement allows a user to decide whether to reset the timer upon completion of a workout or exercise session, or in order to break a workout into smaller segments. The virtual timer may then be reset with the mobile computing device, if the virtual timer is selected to be reset by the mobile computing device. In this way, the present invention may facilitate adjustments to a workout or exercise routine as desired.

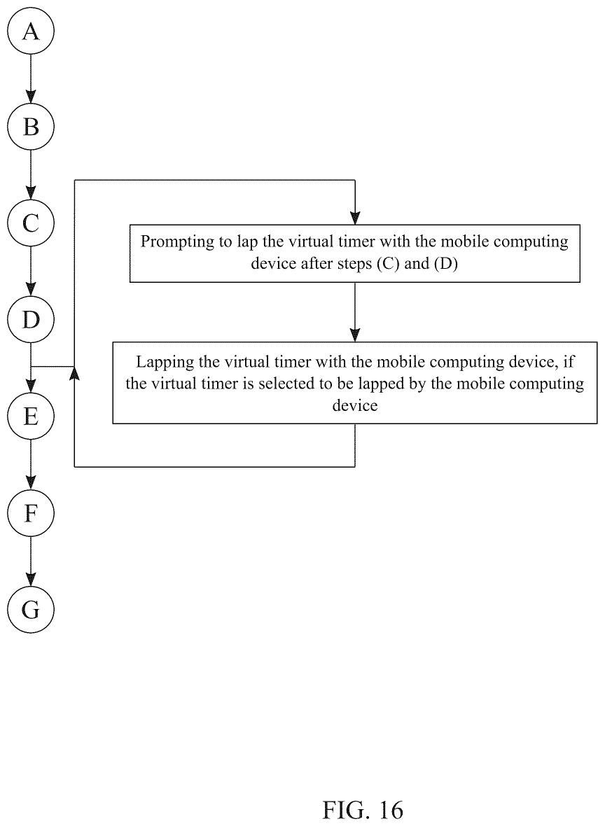

Furthermore, a user may benefit from the ability to create multiple timestamps during a workout, thereby allowing the user to track progress at checkpoints. Therefore, the user may be prompted to lap the virtual timer with the mobile computing device after Steps C and D, as represented in FIG. 16. In this way, a user may decide to generate a time checkpoint in order to break a workout into smaller pieces or to track individual components of a multi-part workout. The virtual timer may then be lapped with the mobile computing device, if the virtual timer is selected to be lapped by the mobile computing device. This allows the present invention to break a larger workout into smaller pieces for subsequent review and analysis by the user.

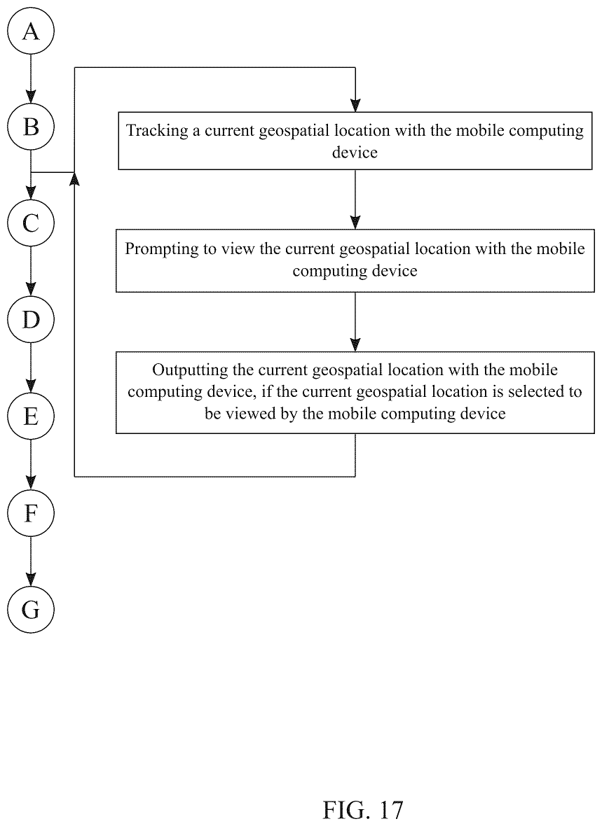

A user may wish to view their location and progress during a workout. To allow for this, a current geospatial location may be tracked with the mobile computing device, as represented in FIG. 17. The GPS module may be used to collect geospatial coordinates for the mobile computing device during the workout. The user may next be prompted to view the current geospatial location with the mobile computing device. In this way, the user may determine whether to view a map or other visual indicator representing distance traveled during a workout. Finally, the current geospatial location may be outputted with the mobile computing device, if the current geospatial location is selected to be viewed by the mobile computing device. The current geospatial location may be presented in any of a variety of different informative formats capable of communicating all relevant information to the user.

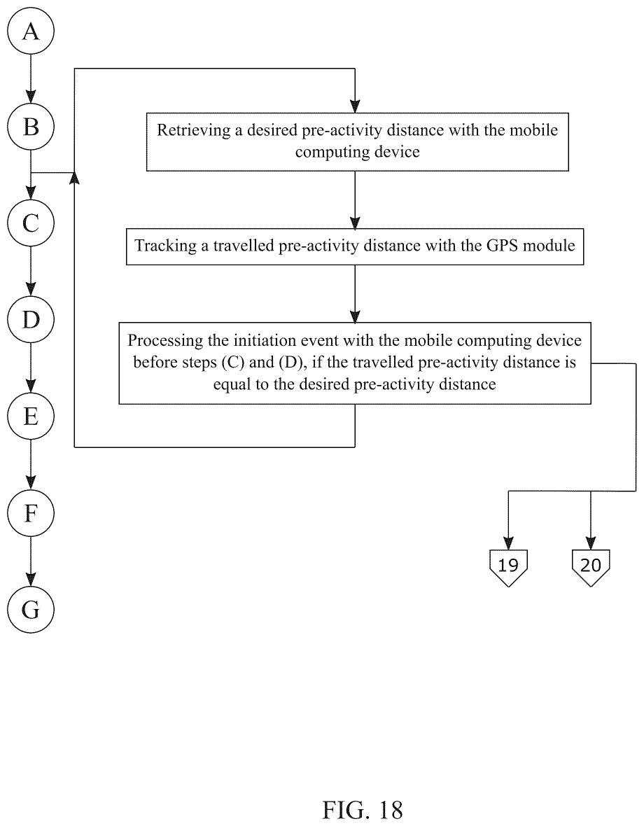

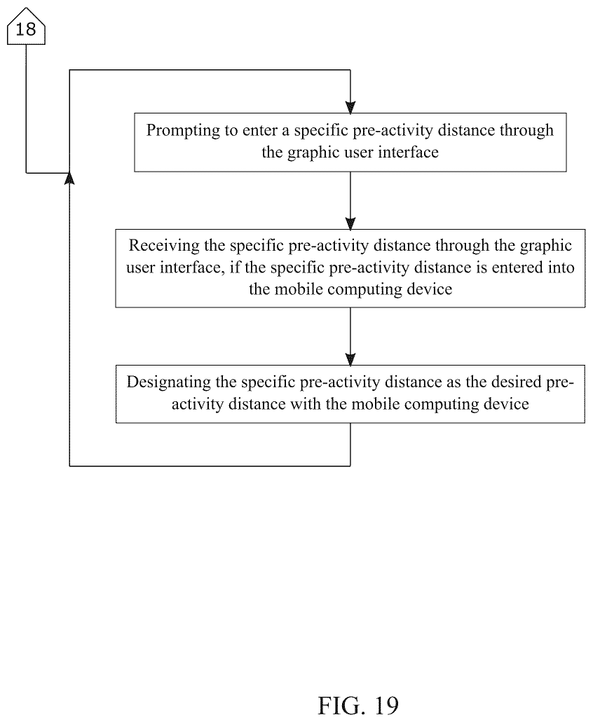

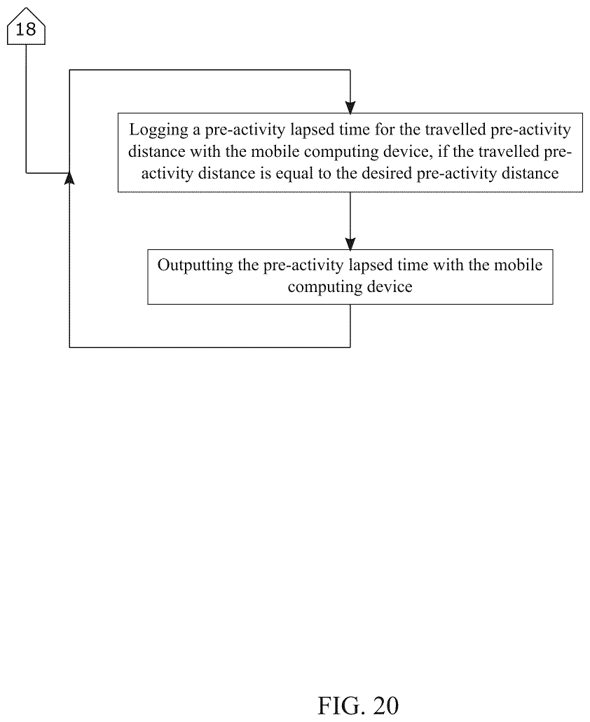

As can be seen in FIG. 18, a user may also want to do a moving warm-up before starting their timed workout (e.g., jogging before starting to run during the timed workout). The mobile computing device can retrieve a desired pre-activity distance from a user, which is a distance that is selected by the user for their moving warm-up. The GPS module is used to track a travelled pre-activity distance, which is the distance that a user physically travels during their warm-up. Thus, when the travelled pre-activity distance is equal to the desired pre-activity distance, the initiation event is processed with the mobile computing device before Steps C and D (e.g., when a user has jogged their pre-set distance, the user is cued by their smart-phone to start running for their workout). Moreover, as can be seen in FIG. 19, in order for a user to select how long their moving warm-up is, a specific pre-activity distance is prompted through the graphic user interface. The specific pre-activity distance is received through the graphic user interface, if the specific pre-activity distance is entered into the mobile computing device, which consequently allows the mobile computing device to designate the specific pre-activity distance as the desired pre-activity distance. Furthermore, as can be seen in FIG. 20, in order for a user to know how long their moving warm-up was, a pre-activity lapsed time for the travelled pre-activity distance is logged with the mobile computing device, when the travelled pre-activity distance is equal to the desired pre-activity distance. A user is eventually notified of the time that it took to complete their moving warm-up by outputting the pre-activity lapsed time with the mobile computing device. In the preferred embodiment, the desired pre-activity distance, the travelled pre-activity distance, and the pre-activity lapsed time are further amended into the athlete database with the remote server.

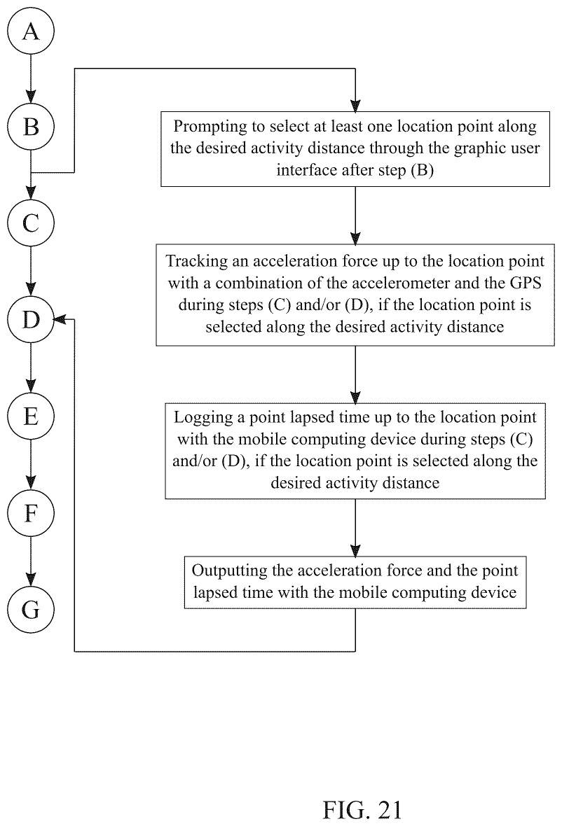

As can be seen in FIG. 21, a user may also want to track an acceleration and a lapsed time for a specific point along a path travelled during their workout. The graphic user interface can prompt a user to select at least one location point along the desired activity distance after Step B. The location point is used to break up a path travelled by the user during their workout so that the user can review and understand their workout in sections instead of an entire traveled path. Moreover, the at least one location point can be a single location point or multiple location points based on a user's preference. An acceleration force up to the location point is tracked with a combination of the accelerometer and the GPS during Steps C and/or D, if the location point is selected along the desired activity distance. A point lapsed time up to the location point is also logged with the mobile computing device during Steps C and/or D, if the location point is selected along the desired activity distance. These steps eventually allow the acceleration force and the point lapsed time to be outputted with the mobile computing device in order to be viewed by a user.

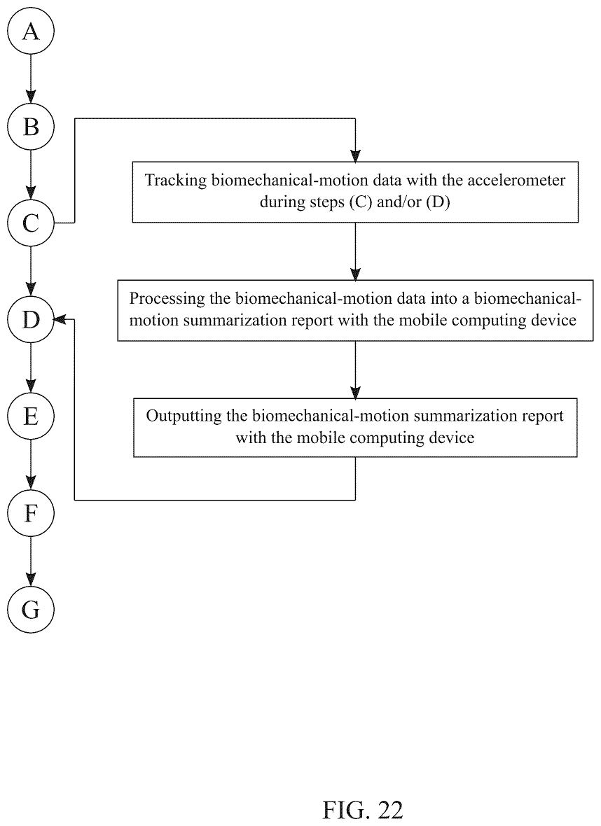

As can be seen in FIG. 22, a user may also want to track biomechanical aspects of their workout. Thus, biomechanical-motion data can be tracked with an accelerometer of the mobile computing device during Step C and/or D. The biomechanical-motion data can be, but is not limited to, overall speed, stride length, arm-swinging motion (e.g., a user holds their smart-phone in their left hand, or the user wears their smart-watch around their left wrist, and, thus, an arm-swinging motion is tracked for the user's left arm), or a combination thereof. The biomechanical-motion data is then compiled into a biomechanical-motion summarization report with the mobile computing device (e.g., the biomechanical-motion summarization report would analyze the intensity of a user swinging their arm). The biomechanical-motion summarization report processes the biomechanical-motion data so that the biomechanical-motion data can easily be interpreted by a user. The biomechanical-motion summarization report is eventually outputted with the mobile computing device in order to be viewed by a user. In the preferred embodiment, the biomechanical-motion data is further amended into the athlete database with the remote server.

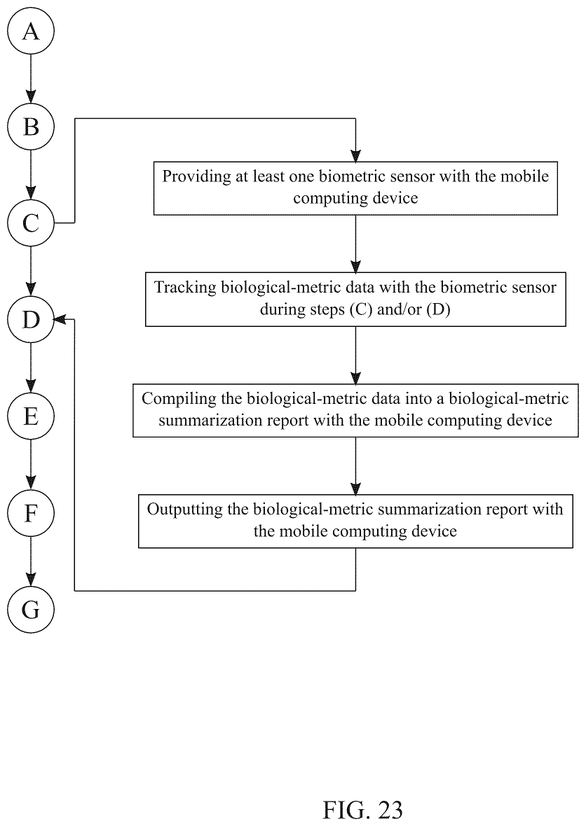

As can be seen in FIG. 23, a user may also want to track biometric aspects of their workout. Thus, biological-metric data can be tracked with at least one biometric sensor of the mobile computing device during Step C and/or D. The biological-metric data can be, but is not limited to, heartrate, calories burnt, or a combination thereof. The biological-metric data is then compiled into a biological-metric summarization report with the mobile computing device. The biological-metric summarization report processes the biological-metric data so that the biological-metric data can easily be interpreted by a user. The biological-metric summarization report is eventually outputted with the mobile computing device in order to be viewed by a user. In the preferred embodiment, the biological-metric data is further amended into the athlete database with the remote server.

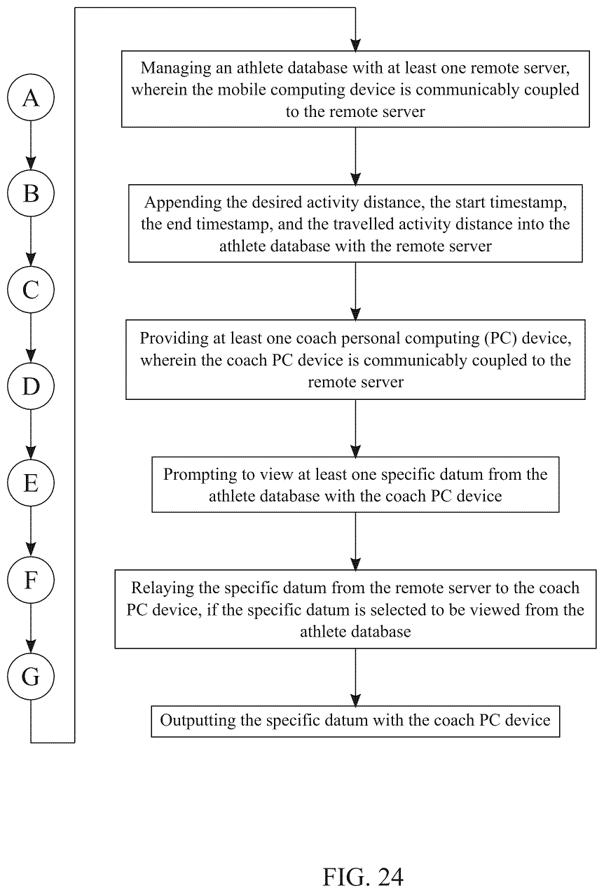

As can be seen in FIG. 24, another user that is not working out with the mobile computing device may want to review metrics and/or information about a user holding the mobile computing device and performing the workout. At least one coach personal computing (PC) device allows this kind of administrative user (e.g., a coach) to view the athlete database in real-time or at a later date, wherein the coach PC device is communicably coupled to the remote server. The coach PC device can be, but is not limited to, a computerized mobile phone (i.e., a smart-phone), a desktop, a laptop, or a tablet PC. The coach PC device prompts an administrative user to view at least one specific datum from the athlete database, and the specific datum is any metric and/or piece of information logged about a user's workout. The specific datum is then relayed from the remote server to the coach PC device, if the specific datum is selected by the administrative user to be viewed from the athlete database. The specific datum is finally outputted with the coach PC device.

Although the invention has been explained in relation to its preferred embodiment, it is to be understood that many other possible modifications and variations can be made without departing from the spirit and scope of the invention as hereinafter.

What is claimed is:

1. A method of timing personal physical activity comprising the steps of:
(A) providing a virtual timer managed by a mobile computing device, wherein the mobile computing device includes a global positioning system (GPS) module, and wherein a graphic user interface is hosted by the mobile computing device;
(B) retrieving a desired activity distance with the mobile computing device;
(C) logging a start timestamp from the virtual timer with the mobile computing device, if an initiation event is processed with the mobile computing device;
(D) tracking a travelled activity distance with the GPS module, if the initiation event is processed with the mobile computing device;
(E) logging an end timestamp from the virtual timer with the mobile computing device, if the travelled activity distance is greater than or equal to the desired activity distance;
(F) designating a time difference between the end timestamp and the start timestamp as an activity lapsed time with the mobile computing device;
(G) outputting the activity lapsed time with the mobile computing device;
sequentially executing steps (B) through (G);
managing an athlete database with at least one remote server, wherein the mobile computing device is communicably coupled to the remote server;
appending the desired activity distance, the start timestamp, the end timestamp, and the travelled activity distance into the athlete database with the remote server;
providing the mobile computing device with at least one accelerometer;
prompting to enter a specific time period for a countdown through the graphic user interface;
setting the specific time period for the countdown with the mobile computing device, if the specific time period is entered through the graphic user interface;
initiating the countdown with the mobile computing device, if the initiation event is processed with the mobile computing device;
outputting the countdown and an end of the countdown with the mobile computing device before steps (C) and (D);
outputting a false start notification with the mobile computing device, if a false start motion is detected with the accelerometer before the end of the countdown, or if the false start motion is detected within a standardized time threshold after the initiation event, wherein the false start notification is a combination of audio, visual, and tactile output by the mobile computing device;
tracking a current geospatial location with the GPS module;
prompting to view the current geospatial location through the graphic user interface;
outputting the current geospatial location with the mobile computing device, if the current geospatial location is selected to be viewed by the mobile computing device, wherein the current geospatial location is visually displayed on a virtual geospatial map through the graphic user interface; and
further appending the false start motion into the athlete database with the remote server.

2. The method as claimed in claim 1 comprising the steps of:
prompting to enter a specific activity distance through the graphic user interface;
receiving the specific activity distance through the graphic user interface, if the specific distance is entered into the mobile computing device; and
designating the specific activity distance as the desired activity distance with the mobile computing device during step (B).

3. The method as claimed in claim 1 comprising the steps of:
retrieving a desired pre-activity distance with the mobile computing device;
tracking a travelled pre-activity distance with the GPS module; and
processing the initiation event with the mobile computing device before steps (C) and (D), if the travelled pre-activity distance is equal to the desired pre-activity distance; and
further appending the desired pre-activity distance and the travelled pre-activity distance into the athlete database with the remote server.

4. The method as claimed in claim 3 comprising the steps of:
prompting to enter a specific pre-activity distance through the graphic user interface;
receiving the specific pre-activity distance through the graphic user interface, if the specific pre-activity distance is entered into the mobile computing device; and
designating the specific pre-activity distance as the desired pre-activity distance with the mobile computing device.

5. The method as claimed in claim 3 comprising the steps of:
logging a pre-activity lapsed time for the travelled pre-activity distance with the mobile computing device, if the travelled pre-activity distance is equal to the desired pre-activity distance; and
outputting the pre-activity lapsed time with the mobile computing device.
further appending the pre-activity lapsed time into the athlete database with the remote server.

6. The method as claimed in claim 1, wherein the standardized time threshold is up to 100 milliseconds after the initiation event.

7. The method as claimed in claim 1 comprising the steps of:
prompting to start the virtual timer through the graphic user interface; and
processing the initiation event with the mobile computing device before steps (C) and (D), if the virtual timer is selected to be started by the mobile computing device.

8. The method as claimed in claim 1 comprising the step of:
processing the initiation event with the mobile computing device before steps (C) and (D), if a spatial motion from rest is detected by the accelerometer.

9. The method as claimed in claim 8 comprising the steps of:
logging a reaction time for the spatial motion from rest with the mobile computing device, if the spatial motion from rest is detected by the accelerometer;
outputting the reaction time with the mobile computing device; and
further appending the reaction time into the athlete database with the remote server.

10. The method as claimed in claim 8 comprising the steps of:
logging an acceleration force for the spatial motion from rest with the mobile computing device, if the spatial motion from rest is detected by the accelerometer;
outputting the acceleration force with the mobile computing device; and
further appending the acceleration force into the athlete database with the remote server.

11. The method as claimed in claim 1 comprising the steps of:
providing a motion threshold stored on the mobile computing device; and
processing the initiation event with the mobile computing device before steps (C) and (D), if a spatial motion above the motion threshold is detected by the accelerometer.

12. The method as claimed in claim 11 comprising the steps of:
logging a reaction time for the spatial motion above the motion threshold with the mobile computing device, if the spatial motion above the motion threshold is detected by the accelerometer;
outputting the reaction time with the mobile computing device; and
further appending the reaction time into the athlete database with the remote server.

13. The method as claimed in claim 11 comprising the steps of:
logging an acceleration force for the spatial motion above the motion threshold with the mobile computing device, if the spatial motion above the motion threshold is detected by the accelerometer;
outputting the acceleration force with the mobile computing device; and
further appending the acceleration force into the athlete database with the remote server.

14. The method as claimed in claim 1 comprising the steps of:
prompting to select at least one location point along the desired activity distance through the graphic user interface after step (B);
tracking an acceleration force up to the location point with a combination of the accelerometer and the GPS during steps (C) and/or (D), if the location point is selected along the desired activity distance;
logging a point lapsed time up to the location point with the mobile computing device during steps (C) and/or (D), if the location point is selected along the desired activity distance; and
outputting the acceleration force and the point lapsed time with the mobile computing device.

15. The method as claimed in claim 1 comprising the steps of:
tracking biomechanical-motion data with the accelerometer during steps (C) and/or (D);
processing the biomechanical-motion data into a biomechanical-motion summarization report with the mobile computing device;
outputting the biomechanical-motion summarization report with the mobile computing device; and
further appending the biomechanical-motion data into the athlete database with the remote server.

16. The method as claimed in claim 1 comprising the steps of:
providing at least one biometric sensor with the mobile computing device;
tracking biological-metric data with the biometric sensor during steps (C) and/or (D);
compiling the biological-metric data into a biological-metric summarization report with the mobile computing device;
outputting the biological-metric summarization report with the mobile computing device; and
further appending the biological-metric data into the athlete database with the remote server.

17. The method as claimed in claim 1 comprising the steps of:
prompting to pause the virtual timer through the graphic user interface after steps (C) and (D); and
pausing the virtual timer with the mobile computing device, if the virtual timer is selected to be paused through the graphic user interface.

18. The method as claimed in claim 1 comprising the steps of:
prompting to reset the virtual timer through the graphic user interface after steps (C) and (D); and
resetting the virtual timer with the mobile computing device, if the virtual timer is selected to be reset through the graphic user interface.

19. The method as claimed in claim 1 comprising the steps of:
providing at least one coach personal computing (PC) device, wherein the coach PC device is communicably coupled to the remote server;
prompting to view at least one specific datum from the athlete database with the coach PC device;
relaying the specific datum from the remote server to the coach PC device, if the specific datum is selected to be viewed from the athlete database; and
outputting the specific datum with the coach PC device.
